(19) United States
(12) Patent Application Publication
(10) Pub. No.: US 2003/0039709 A1
(43) Pub. Date: Feb. 27, 2003

(54) Composition including essential oils

(76) Inventor: W. E. Thrash, Tyler, TX (US)

Correspondence Address: Ronald B. Sefrna, SEFRNA & ASSOCIATES, 505 South Bois D'Arc, P.O. Box 567, Tyler, TX, 75710-0567, US

(21) Appl. No.: 10/224,974

(22) Filed: Aug. 20, 2002

Related U.S. Application Data
(60) Provisional application No. 60/313,480, filed on Aug. 20, 2001.

(51) Int. Cl. A61K 35/78

(52) U.S. Cl. 424/735; 424/736; 424/744

(57) ABSTRACT

A composition for application to the skin includes a combination of essential oils consisting essentially of sweet almond essential oil, sweet orange essential oil, and lemon essential oil, and provides unexpectedly beneficial and effective treatment results. The proportion of each essential oil within the combination of essential oils ranges between about ten percent and about ninety percent. The essential oils are preferably suspended with moisturizing agents in water by an emulsifying agent to form a lotion or gel base to facilitate application to the skin. Aloe Vera extract and vitamin E may also be added.

### FIELD OF THE INVENTION

The present invention generally relates to compositions of matter, and more specifically relates to compositions including essential oils, principally for cosmetic uses as shaving gels and lotions and skin lotions.

### BACKGROUND OF THE INVENTION

Essential oils are natural components of plants that are well known to mankind, and have been extracted and used for centuries to provide fragrance to a wide variety of preparations and formulations, including perfumes, cosmetics, and foods. Several essential oils, specifically menthol, eucalyptus oil, camphor, peppermint oil, and wintergreen oil, are used in topical preparations for pain relief. Essential oils are used in these preparations in high concentrations, up to about 30% of the preparation. Vapors from some essential oils are believed to be beneficial when inhaled, as in “aroma therapy”, for the relief of various conditions.

The use of essential oils known in the prior art for cosmetic products have been primarily for the purpose of fragrance or flavor to the products, rather than for any direct effect or benefit derived from the essential oils themselves. The properties of, and attributed to, specific essential oils and various combinations of essential oils have been studied and cataloged for many years, but the vast number of possible combinations has not been fully explored.

### SUMMARY OF THE INVENTION

The present invention discloses a composition of essential oils that, in combination, has been found to have properties and benefits in certain cosmetic applications that are unexpected and unanticipated from the known properties of the constituent essential oils. Although the mechanism by which the unexpected, synergistic effect of the composition of the invention is not fully understood, the effectiveness has been clearly demonstrated.

The composition of the invention consists essentially of a mixture of the following three essential oils: sweet almond oil, sweet orange oil, and lemon oil. The three essential oils are preferably combined with moisturizers, emulsifying agents, and other carriers to form a lotion or gel base to facilitate application to the skin. The carrier lotion or gel is a “vanishing” type carrier, with some components that evaporate and some that are absorbed into the skin, carrying the essential oils to and into the surface of the skin. Although the unusual and unexpected effects resulting from the invention are achieved by the combination of essential oils alone, an further effect can be achieved by the addition of a concentrate of aloe vera to the lotion.

### DETAILED DESCRIPTION OF THE INVENTION

In a particularly preferred composition, these three essential oils making up the synergistic combination are present in the following relative percentages:

| | |
|---|---|
| sweet almond oil | 26.4% |
| sweet orange oil | 36.8% |
| lemon oil | 36.8% |

Although a combination of essential oils in these specific proportions has been found to be particularly effective in a preparation for use as a shaving gel or lotion, it is to be clearly understood that the invention is not limited to the combination of the identified essential oils in this specific proportion. The synergistic effect of the combination of the three disclosed essential oils is exhibited by compositions in which each is present in a range of proportions, both lower and higher than in the cited example. The synergistic effect of the combination of the three essential oils in the composition of the invention may be achieved when each constituent of the composition is present in a concentration ranging from about 10% to about 90%, with a concentration ranging from about 20% to about 50% being preferred.

Preparations including the composition of essential oils disclosed by the invention have been formulated and found to be unexpectedly effective and beneficial for cosmetic uses including preparation of skin and hair for shaving, soothing of the skin following shaving, smoothing and moisturizing the skin, soothing the effects of skin irritation, and enhancing the suppleness and elasticity of the skin. In general, the composition of essential oils of the invention has been found to provide the unexpected results that have been experienced when used in a lotion or gel formulation in proportions ranging from about 1% to about 5% of the overall formulation.

In one example, a formulation of the following composition was prepared, with the proportions expresses in percent by weight:

| | |
|---|---|
| deionized water | 96.88% |
| methylparaben | .12% |
| glycerin | .20% |
| Carbopol Ultrez 10 ® polymer | .50% |
| triethanolamine | .35% |
| sweet almond oil | .50% |
| sweet orange oil | .70% |
| lemon oil | .70% |
| Aloe Vera concentrate (44X) | .05% |

The particular formulation of this example was prepared for evaluation as a shaving lotion. As will be understood b) persons knowledgeable in the cosmetics field, the combination of the constituents of the formulation, with the exception of the composition of essential oils of the invention, is not unique or unusual, and would be expected to provide a lubricating and moisturizing effect for the skin to at least some degree. However, in test uses the formulation, with the inclusion of the composition of essential oils of the invention, was found to provide results and benefits far exceeding any expectation based on the other constituents of the formulation or on known properties of any essential oils.

Initial test subjects included men with heavy facial beards, who typically experienced skin irritation when shaving, and who further typically found it necessary to shave twice a day in order to maintain a smoothly shaven appearance, regardless of the shaving technique or products previously used. All test subjects uniformly reported that use of the formulation of the example resulted in closer shaves with little or no irritation (substantially less than previously experienced), and a reduction in frequency of shaving needed to maintain the same appearance.

Most significantly, and most unexpectedly, African American men who had previously experienced severe problems and discomfort from shaving because of “razor bumps” or “ingrown beards” reported an unexpected alleviation of the problem and relief of shaving discomfort from use of the formulation of the example. The mechanism for the unexpected and unpredicted effectiveness of the composition of the invention in alleviating these problems is not well understood, but the favorable results reported by test subjects are universal.

In another example, a formulation with a somewhat different proportional relationship among constituent ingredients was prepared, as follows:

| | |
|---|---|
| deionized water | 97.00% |
| methylparaben | .20% |
| glycerin | .20% |
| Carbopol Ultrez 10 ® polymer | .50% |
| triethanolamine | .35% |
| sweet almond oil | .50% |
| sweet orange oil | .70% |
| lemon oil | .45% |
| Aloe Vera concentrate (44X) | .05% |
| Vitamin E | .05% |

This formulation was tested in the same manner as the formulation given in the previous example, and provided the same previously unexpected results and benefits. The effectiveness of the formulated for alleviating “razor bumps” among African American males was pronounced, and, again, completely unexpected from the body of prior art relating to essential oils and combinations thereof. The addition of Vitamin E has no apparent involvement in the synergistic effect of the combination of the essential oils, but may be a desirable ingredient because of its known beneficial effects on the skin. The proportion of Vitamin E may be varied within the same general range as the proportion of Aloe Vera.

As can be generally seen from the foregoing examples, the proportion of the combination of the essential oils of the invention to the formulation as a whole is preferred to be within the range of about 1% to about 3%, with about 2% being particularly preferred.

Although the synergistic effect of the composition of essential oils of the invention is not dependent upon the inclusion of Aloe Vera concentrate or extract, the effect may be enhanced by the inclusion of a relatively low proportion of that constituent with the composition of essential oils, as in the cited example. In an alternative embodiment of the composition of the invention, the composition includes Aloe Vera concentrate with the combination of essential oils in a proportion relative to the essential oils ranging from about 1% to about 20%, with the range of about 2% to about 5% being preferred.

The foregoing description of the composition of the invention, the disclosure of a presently particularly preferred composition, the recitation of an example of a formulation including the composition of the invention, and the disclosure of an alternative composition are all intended to be illustrative, and by no means limiting of the full scope of the invention.

1. A cosmetic composition of essential oils for topical application to the skin, consisting of sweet almond essential oil, sweet orange essential oil, and lemon essential oil.

2. The composition of essential oils of claim 1, wherein the proportion of each of said essential oils in said composition is within the range of about ten percent to about ninety percent.

3. The composition of essential oils of claim 1, wherein the proportion of each of said essential oils in said composition is within the range of about twenty percent to about fifty percent.

4. The composition of essential oils of claim 1, wherein the proportion of said sweet almond essential oil in said composition is within the range of about twenty percent to about thirty percent, wherein the proportion of said sweet orange essential oil is within the range of about thirty percent to about forty percent, and wherein the proportion of said lemon essential oil is within the range of about thirty percent to about forty percent.

5. The composition of essential oils of claim 1, wherein the proportion of said sweet orange essential oil is approximately equal to the proportion of said lemon essential oil in said composition.

6. The composition of essential oils of claim 5, wherein the proportion of said sweet orange oil and the proportion of said lemon oil are approximately 1.25 to 1.75 times the proportion of said sweet almond oil.

7. The composition of essential oils of claim 1, wherein said essential oils are combined with concentrated aloe vera extract.

8. The combination of claim 7, wherein the proportion of said concentrated aloe vera extract in said combination is within the range of about one percent to about ten percent.

9. The combination of claim 7, wherein the proportion of said concentrated aloe very extract in said combination is within the range of about two percent to about five percent.

10. A cosmetic composition for application to the skin, comprising
a combination of essential oils consisting of sweet almond essential oil, sweet orange essential oil, and lemon essential oil;
a solvent;
a moisturizing agent for moisturizing the skin; and
an emulsifying agent for forming a uniform suspension of said combination of essential oils and said moisturizing agent in said solvent.

11. The cosmetic composition of claim 10, wherein said solvent is water.

12. The cosmetic composition of claim 11, wherein said emulsifying agent is an organic polymer.

13. The cosmetic composition of claim 10, wherein said moisturizing agent comprises glycerin.

14. The cosmetic composition of claim 10, further comprising aloe vera extract.

15. The cosmetic composition of claim 10, wherein said combination of essential oils comprises about one percent to about five percent of said composition, by weight.

16. The cosmetic composition of claim 14, wherein said aloe vera extract is in concentrated form, and wherein said aloe vera extract comprises about five one hundredths of one percent to about one percent of said composition, by weight.

17. The cosmetic composition of claim 10, wherein said combination of essential oils comprises between about one percent and about three percent of said composition, by weight.

18. The cosmetic composition of claim 10, wherein said combination of essential oils comprises about two percent of said composition, by weight.

19. The cosmetic composition of claim 10, wherein the proportion of each of said essential oils in said combination of essential oils is within the range of about ten percent to about ninety percent.

20. The cosmetic composition of claim 10, wherein the proportion of each of said essential oils in said combination of essential oils is within the range of about twenty percent to about fifty percent.
